(19) United States
(12) Patent Application Publication
(10) Pub. No.: US 2015/0246027 A1
(43) Pub. Date: Sep. 3, 2015

(54) FIXED DOSE PHARMACEUTICAL COMPOSITION COMPRISING DEFERASIROX AND DEFERIPRONE

(71) Applicants: Emily COTTRILL, (US); CIPLA LIMITED, Maharashtra (IN)

(72) Inventors: Shrinivas Madhukar Purandare, Mumbai (IN); Geena Malhotra, Mumbai (IN)

(73) Assignee: Cipla Limited, Mumbai (IN)

(21) Appl. No.: 14/430,063

(22) PCT Filed: Nov. 12, 2013
(86) PCT No.: PCT/GB2013/000481

§ 371 Date: Mar. 20, 2015

(30) Foreign Application Priority Data
Nov. 12, 2012 (IN) 3290/MUM/2012

(51) Int. Cl. A61K 31/4412; A61K 45/06; A61K 31/4196; A61K 9/20

(52) U.S. Cl. CPC A61K 31/4412; A61K 9/2095; A61K 45/06; A61K 31/4196

(57) ABSTRACT

The present invention relates to a fixed dose pharmaceutical composition comprising iron chelating agents.

### FIELD OF INVENTION

The present invention relates to a fixed dose pharmaceutical composition comprising iron chelating agents, a process for preparing such a pharmaceutical composition and the use of the said pharmaceutical composition for the treatment of chronic iron overload.

### BACKGROUND AND PRIOR ART

Thalassemias are inherited autosomal recessive disorders characterised by the reduced rate of hemoglobin synthesis due to a defect in the α or β globin chain synthesis.

Chronic iron overload occurs commonly in patients with beta-thalassemia major (TM) mainly due to frequent blood transfusions that are carried out at the rate of approximately 0.5 mg/kg body weight per day for the management of several conditions including β-thalassemia, sickle cell disease and myelodysplastic syndromes.

Each unit of blood contains iron and since the human body has no physiological mechanism to actively excrete the excess iron, repeated blood transfusions result in excessive accumulation of iron. This excess of iron deposited in body tissues can cause severe damage to vital organs such as liver, heart and the endocrine organs such as the hypothalamus, pituitary, thyroid and parathyroid glands as well as the gonads. This may ultimately lead to many complications including cardiomyopathy, liver cirrhosis and diabetes mellitus and eventually reduced life expectancy.

Chelation therapy is generally started when transfusion dependent patients have received 10-12 transfusions or when the ferritin is constantly greater than 1000 mg/L. Ferritin is a ubiquitous intracellular protein that stores iron and releases it in a controlled fashion. The amount of ferritin stored reflects the amount of iron stored. Serial ferritin levels provide better indication of iron loading as compared to random ferritin measurement. Compliance with chelation therapy is a major predictor of long-term morbidity and mortality. Current options for chelation are desferrioxamine, deferiprone and deferasirox and in some cases combinations of chelating agents. Desferrioxamine has been the most widely used chelator though there is increasing experience with deferiprone and deferasirox.

Deferasirox is an orally active iron chelator and has been approved for the treatment of iron overload in transfusion dependent anemia's (transfusion hemosiderosis) particularly thalassemia major, thalassemia intermediate and in sickle cell disease to reduce iron-related morbidity and mortality in patients having an age of two years and older.

Deferasirox has the chemical name 4-[3,5-bis(2-hydroxyphenyl)-[1,2,4]triazol-1-yl]enzoic acid, and is reported to have the following chemical structure.

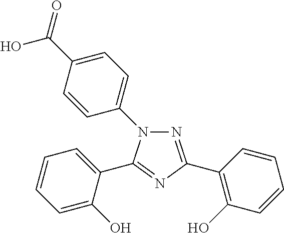 [SMILES: O=C(O)c1ccc(-n2nc(-c3ccccc3O)nc2-c2ccccc2O)cc1]

Deferasirox mobilizes tissue iron by forming soluble stable complexes that are then excreted in the faeces. It is a tridentate iron chelator requiring two molecules of the drug to form a stable complex. Iron is chelated both from the reticuloendothelial cells (RE cells) as well as various parenchymal tissues. The chelated iron is cleared by the liver and excreted through the bile. It also has the ability to prevent the myocardial cell iron uptake by removing iron directly from myocardial cells.

Deferasirox is commercially available as a dispersible tablet (EXJADE®) for oral administration. EXJADE® is supplied as a dispersible tablet containing 125 mg, 250 mg and 500 mg of deferasirox per tablet. Deferasirox is administered as a once daily oral iron chelator, which is prescribed as a dispersible tablet, i.e., a tablet which needs to be dispersed in an aqueous medium prior to administration.

Deferasirox is highly water-insoluble and is highly lipid-soluble, and is also observed to possess good permeability. According to the Bio-pharmaceutics Classification System (BCS), it has been classified as a Class II drug, implying that it is a poorly soluble and a highly permeable drug. Though deferasirox is highly water-insoluble, whatever limited solubility it has, that too exhibits a high pH-dependent solubility. Though it is practically insoluble in lower pH, even at a pH of 6.8, it still remains insoluble, until the buffer strength is altered to get optimal dissolution profile.

Deferasirox being practically insoluble in aqueous media exhibits a generally poor dissolution profile and hence consequently poor bioavailability.

Deferiprone, chemically known as (3-hydroxy-1,2-dimethylpyridin-4-one) is an orally active, iron-chelating agent and is represented by the following chemical structure.

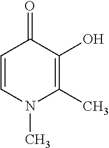

Deferiprone exhibits an affinity for ferric ion (iron III). Deferiprone binds with ferric ions to form neutral 3:1 (deferiprone:iron) complexes that are stable over a wide range of pH values.

Deferiprone is indicated for the treatment of patients with transfusional iron overload due to thalassemia syndromes when current chelation therapy is inadequate. The recommended initial dose of Deferiprone is 25 mg/kg, orally, three times per day for a total of 75 mg/kg/day. The maximum dose is 33 mg/kg, three times per day for a total of 99 mg/kg/day.

Deferiprone is commercially available as FERRIPROX® for oral administration and are supplied as 500 mg film coated capsule-shaped tablets.

Deferiprone is sparingly soluble in water (12.95 mg/ml) and, less than 10% of the ingested dose is excreted as the active form, the majority of the drug being excreted as the inactive glucuronide metabolite.

WO2004035026 discloses a dispersible tablet of deferasirox wherein the active ingredient is present in an amount ranging from 5% to 40% by weight based on the total weight of the tablet.

WO2005097062 discloses a dispersible tablet of deferasirox wherein the active ingredient is present in an amount ranging from 42% to 65% by weight based on the total weight of the tablet.

WO2007045445 discloses a dispersible tablet of deferasirox or a pharmaceutically acceptable salt thereof present in an amount ranging from 42% to 65% by weight based on the total weight of the tablet and at least one pharmaceutically acceptable excipient suitable for the preparation of dispersible tablets and the process for making said dispersible tablet.

WO2009067557 discloses a process of preparing deferasirox formulations having sufficiently high dissolution rate and good bioavailability wherein the said process comprises co-milling deferasirox with at least two pharmaceutically acceptable excipients in the absence of any solvent.

WO2010035282 discloses an oral pharmaceutical composition comprising deferasirox in the form of a dispersible tablet wherein the active ingredient has a mean particle size less than about 100 μm and is present in an amount greater than 66% by weight based on total weight of the tablet.

WO2012042224 discloses pharmaceutical compositions comprising deferasirox in the form of particles, wherein the particles have an average particle size of less than or equal to about 2000 nm.

WO2009129592 discloses a bitter and palatable oral liquid formulation of deferiprone and a taste masking composition comprising a sweetener, thickening and suspension aid, humectants and an flavoring agent.

WO2001049266 discloses an iron chelator delivery system comprising an iron chelator and a lipid carrier.

“Successful chelation therapy with the combination of deferasirox and deferiprone in a patient with thalassemia major and persisting severe iron overload after single-agent chelation therapies' 2011, British Journal of Haematology, 154, 654-665. This article highlights the concerns regarding the compliance issues of deferoxamine. The article further states that the efficacy and safety of the combination of deferiprone and deferasirox in a trial may determine whether this combination could be a useful option in tailoring individual chelation therapy for thalassemia major (TM) patients with iron overload.

The above documents disclose deferasirox and deferiprone separately in various types of pharmaceutical formulations, but it has been observed that there still exist problems with such formulations, particularly in terms of the high frequency of administration, associated patient compliance problems and also the overall cost of the medication.

These prior arts do not reveal any compositions in the form of a fixed dose which is administered as a single unit dose or single dosage form. Envisaging the problems of the prior art, the inventors of the present invention have developed a pharmaceutical composition which eliminates or substantially minimises these problems, leading to improved patient compliance, a reduction in the frequency of administration, and also reduced medication costs.

### OBJECT OF THE INVENTION

An object of the present invention is to provide a fixed dose pharmaceutical composition comprising iron chelating agents.

Another object of the present invention is to provide a fixed dose pharmaceutical composition comprising iron chelating agents optionally with one or more pharmaceutically acceptable excipients.

Yet another object of the present invention is to provide a fixed dose pharmaceutical composition comprising iron chelating agents to ensure patient compliance.

Another object of the present invention is to provide a fixed dose pharmaceutical composition comprising iron chelating agents having improved surface area and solubility.

Another object of the present invention is to provide a fixed dose pharmaceutical composition comprising iron chelating agents having synergistic effect.

Another object of the present invention is to provide a fixed dose pharmaceutical composition comprising iron chelating agents having a reduced dose.

Another object of the present invention is to provide a process for preparing a fixed dose pharmaceutical composition comprising iron chelating agents optionally with one or more pharmaceutically acceptable excipients.

Another object of the present invention is to provide a method of reducing chronic iron overload by administering a fixed dose pharmaceutical composition comprising iron chelating agents.

Another object of the present invention is to provide the use of treating chronic iron overload in thalassemia major patients through chelation therapy by administering a fixed dose pharmaceutical composition comprising iron chelating agents.

### SUMMARY OF THE INVENTION

According to an aspect of the present invention, there is provided a fixed dose pharmaceutical composition comprising at least two iron chelating agents and optionally one or more pharmaceutically acceptable excipients.

According to an aspect of the present invention, there is provided a fixed dose pharmaceutical composition comprising at least two iron chelating agents and optionally one or more pharmaceutically acceptable excipients for use in the treatment of chronic iron overload.

According to an aspect of the present invention, there is provided the use of a fixed dose pharmaceutical composition comprising at least two iron chelating agents and optionally one or more pharmaceutically acceptable excipients in the manufacture of a medicament for treating chronic iron overload.

According to an aspect of the invention, there is provided a method of treating chronic iron overload wherein the method comprises administering a fixed dose pharmaceutical composition comprising at least two iron chelating agents and optionally one or more pharmaceutically acceptable excipients.

According to another aspect of the invention, there is provided a process for the preparation of a fixed dose pharmaceutical composition comprising mixing at least two iron chelating agents and optionally one or more pharmaceutically acceptable excipients.

### DETAILED DESCRIPTION OF THE INVENTION

Patients with thalassemia major accumulate body iron over time as a consequence of continuous red blood cell transfusions which cause hepatic, endocrine, and cardiac complications. Despite the availability of iron chelators, some patients fail to respond adequately to monotherapy with any of them. Combination therapy, consisting in the use of two iron chelators on the same day, has been introduced to increase the efficacy and to induce negative iron balance in patients with severe iron overload. Extensive long-term experience has shown that combined iron chelation therapy rapidly reduces liver iron, serum ferritin, and myocardial siderosis, improves cardiac function, reverses and prevents endocrine complications, reduces cardiac mortality, and improves survival.

Such combination therapy may involve different dosage regimens as well as different frequencies of administration of the iron chelators and thus it generally may result in non-completion of therapy leading to the worsening of underlying disease as a result of such non-compliance, which is especially observed in geriatric and pediatric patients.

Efforts to improve such compliance have been aimed at by simplifying the medication packaging, providing effective medication reminders, improving patient education, and limiting the number of medications prescribed simultaneously. However, such efforts have not been able to completely resolve the drawbacks associated with patient compliance.

The present invention provides a fixed dose pharmaceutical composition comprising iron chelating agents which would ensure patient compliance due to simplification of therapy. The term fixed dose refers to a combination of two active ingredients in a single dosage form or single unit dose.

Iron chelating agents for use in the composition of the invention include, but are not limited to, deferasirox and deferiprone.

The present invention thus provides a fixed dose pharmaceutical composition comprising a combination of deferasirox and deferiprone.

Potential advantages of such fixed dose combinations include an improvement of the benefit/risk assessment due to addition or potentiation of therapeutic activities of their substances, which results in a level of efficacy similar to the one achievable by each active substance used alone at higher doses than in combination. Further, they are associated with a better safety profile or a level of efficacy above the one achievable by a single substance with an acceptable safety profile. Another advantage is the counteracting by one substance of an adverse reaction produced by another one.

It will be understood, however, that specific dose level and frequency of dosage of the combination according to the invention for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The term “Deferasirox” is used in broad sense to include not only “Deferasirox” per se but also its pharmaceutically acceptable derivatives thereof. Suitable derivatives include pharmaceutically acceptable salts, pharmaceutically acceptable solvates, pharmaceutically acceptable hydrates, pharmaceutically acceptable isomers, pharmaceutically acceptable esters, pharmaceutically acceptable anhydrates, pharmaceutically acceptable enantiomers, pharmaceutically acceptable polymorphs, pharmaceutically acceptable prodrugs, pharmaceutically acceptable tautomers and/or pharmaceutically acceptable complexes thereof.

The term “Deferiprone” is used in broad sense to include not only “Deferiprone” per se but also its pharmaceutically acceptable derivatives thereof. Suitable derivatives include pharmaceutically acceptable salts, pharmaceutically acceptable solvates, pharmaceutically acceptable hydrates, pharmaceutically acceptable isomers, pharmaceutically acceptable esters, pharmaceutically acceptable anhydrates, pharmaceutically acceptable enantiomers, pharmaceutically acceptable polymorphs, pharmaceutically acceptable prodrugs, pharmaceutically acceptable tautomers and/or pharmaceutically acceptable complexes thereof.

Preferably, the fixed dose pharmaceutical composition, according to the present invention comprises deferasirox and deferiprone, in their free base forms.

Preferably, the fixed dose pharmaceutical composition, according to the present invention comprises deferasirox and deferiprone, in the ratio of 1:1.5-5.

The term “pharmaceutical composition” includes tablets, soft gelatin capsule, capsules (filled with powders, powders for reconstitution, pellets, beads, mini-tablets, pills, micro-pellets, small tablet units, MUPS, disintegrating tablets, dispersible tablets, granules, sprinkles microspheres and multiparticulates), sachets (filled with powders, pellets, beads, mini-tablets, pills, micro-pellets, small tablet units, MUPS, disintegrating tablets, dispersible tablets, granules, sprinkles microspheres and multiparticulates) and sprinkles, however, other dosage forms such as liquid dosage forms (liquids, liquid dispersions, suspensions, solutions, emulsions, sprays, spot-on), injection preparations, gels, aerosols, ointments, creams, controlled release formulations, lyophilized formulations, delayed release formulations, extended release formulations, pulsatile release formulations, and mixed immediate release and controlled release formulations etc. may also be envisaged under the ambit of the invention.

The fixed dose pharmaceutical composition is presented in the form of oral, parenteral (subcutaneous, intravenous, intramuscular, intradermal, intraperitoneal and the like) or topical (sprays, solutions, suspensions, ointments, drops, in-situ gel, aerosols, ointments, microspheres, creams, gels, patches, films and the like) dosage forms.

Preferably, the fixed dose pharmaceutical composition is presented in the form of oral dosage forms.

Preferably, the fixed dose pharmaceutical composition is a solid oral dosage form. More preferably, the solid dosage form is in the form of tablet, capsule (filled with powders, powders for reconstitution, pellets, beads, mini-tablets, pills, micro-pellets, small tablet units, MUPS, disintegrating tablets, dispersible tablets, granules, sprinkles, microspheres, and multiparticulates), sachets (filled with powders, powders for reconstitution, pellets, beads, mini-tablets, pills, micro-pellets, small tablet units, MUPS, disintegrating tablets, dispersible tablets, granules, sprinkles, microspheres and multiparticulates) and sprinkles. A tablet formulation is the preferred solid dosage form due to its greater stability, less risk of chemical interaction between different medicaments, smaller bulk, accurate dosage, and ease of production.

According to one embodiment, the fixed dose pharmaceutical composition, according to the present invention, may be administered as a multilayer tablet, preferably a bilayer tablet, wherein each layer separately contains a drug and pharmaceutically acceptable excipients which are then compressed to give a bilayer tablet.

According to another embodiment, the fixed dose pharmaceutical composition, according to the present invention, may be administered as granules directly or filled into capsules or sachets.

According to a preferred embodiment, the fixed dose pharmaceutical composition, according to the present invention, may be administered as a dispersible tablet.

The inventors of the present invention have further observed that the solubility properties of iron chelating agents were improved by nanosizing resulting in better solubility thus leading to better bioavailability of the drug.

Nanosization of hydrophobic or poorly water-soluble drugs generally involves the production of drug nanocrystals through either chemical precipitation (bottom-up technology) or disintegration (top-down technology). Different methods may be utilized to reduce the particle size of the hydrophobic or poorly water soluble drugs. [Huabing Chen et al., discusses the various methods to develop nanoformulations in “Nanonization strategies for poorly water-soluble drugs,” Drug Discovery Today, Volume 00, Number 00, March 2010].

The present invention thus provides a fixed dose pharmaceutical composition comprising deferasirox and deferiprone wherein deferasirox and deferiprone have an average particle size of less than or equal to about 2000 nm, preferably less than or equal to about 1000 nm.

The term average particle size as used herein refers to the average diameter of the particles.

Mostly all particles have a particle size of less than or equal to about 2000 nm, preferably less than or equal to about 1000 nm.

The term “particles” as used herein refers to an individual particle of deferasirox, or particles of deferasirox or deferasirox granules or deferasirox compositions and/or mixtures thereof.

The particles of the present invention can be obtained by any of the process such as but not limited to milling, precipitation, homogenization, high pressure homogenization, spray-freeze drying, supercritical fluid technology, double emulsion/solvent evaporation, PRINT (Particle replication in non-wetting templates), thermal condensation, ultrasonication and spray drying.

Accordingly, the process of milling can comprise dispersing drug particles in a liquid dispersion medium in which the drug is poorly soluble, followed by applying mechanical means in the presence of grinding media to reduce the particle size of drug to the desired effective average particle size.

The fixed dose pharmaceutical compositions of the present invention can be manufactured by any of the types of processes as described above.

Preferably, the process comprises (1) homogenizing deferasirox and/or deferiprone and at least one excipient to produce a homogenized dispersion; and
(2) milling the said homogenized dispersion to produce a slurry comprising deferasirox and/or deferiprone particles having an average particle size of less than or equal to about 2000 nm
(3) processing the slurry to obtain the desired dosage form.

Suitable excipients may be used for formulating the various dosage forms according to the present invention.

Surface stabilizers, may be used in compositions of the invention. These are surfactants that are capable of stabilizing the increased surface charge of the drug. Suitable amphoteric, non-ionic, cationic or anionic surfactants may be included in the pharmaceutical composition of the present invention.

Surfactants that can be used in the compositions of the invention maycomprise one or more of, but not limited to Polysorbates, Sodium dodecyl sulfate (sodium lauryl sulfate), Lauryl dimethyl amine oxide, Docusate sodium, Cetyl trimethyl ammonium bromide (CTAB) Polyethoxylated alcohols, Polyoxyethylene sorbitan, Octoxynol, N, N-dimethyldodecylamine-N-oxide, Hexadecyltrimethylammonium bromide, Polyoxyl 10 lauryl ether, Brij, Bile salts (sodium deoxycholate, sodium cholate), Polyoxyl castor oil, Nonylphenol ethoxylate Cyclodextrins, Lecithin, Methylbenzethonium chloride. Carboxylates, Sulphonates, Petroleum sulphonates, alkylbenzenesulphonates, Naphthalenesulphonates, Olefin sulphonates, Alkyl sulphates, Sulphates, Sulphated natural oils & fats, Sulphated esters, Sulphated alkanolamides, Alkylphenols, ethoxylated & sulphated, Ethoxylated aliphatic alcohol, polyoxyethylene surfactants, carboxylic esters Polyethylene glycol esters, Anhydrosorbitol ester & it's ethoxylated derivatives, Glycol esters of fatty acids, Carboxylic amides, Monoalkanolamine condensates, Polyoxyethylene fatty acid amides, Quaternary ammonium salts, Amines with amide linkages, Polyoxyethylene alkyl & alicyclic amines, N,N,N,N tetrakis substituted ethylenediamines 2-alkyl 1-hydroxyethyl 2-imidazolines, N-coco 3-aminopropionic acid/sodium salt, N-tallow 3-iminodipropionate disodium salt, N-carboxymethyl n dimethyl n-9 octadecenyl ammonium hydroxide, n-cocoamidethyl n-hydroxyethylglycine sodium salt or mixtures thereof.

Preferably, the surfactants may be present in an amount ranging from about 0.1% to about 20% by weight of the composition.

Viscosity builders/enhancers/imparting agents are excipients that are capable of stabilizing the formulation by increasing the viscosity of the formulation and thus preventing physical interaction of nanoparticles under the operating conditions employed.

Viscosity builders that can be used in compositions of the invention may comprise one or more, but not limited to derivatives of sugars, such as lactose, sucrose, saccharose, hydrolyzed starch (maltodextrin) or mixtures thereof.

Preferably, the viscosity builders may be present in an amount ranging from about 3% to about 35% by weight of the composition.

Polymers, for use in compositions of the invention, may comprise one or more hydrophilic polymers, but not limited to cellulose derivatives like hydroxypropylcellulose, hydroxymethylcellulose, hydroxypropylmethylcellulose, methylcellulose polymers hydroxyethylcellulose, sodium carboxymethylcellulose, carboxymethylene and carboxymethyl hydroxyethylcellulose; acrylics like acrylic acid, acrylamide, and maleic anhydride polymers, acacia, gum tragacanth, locust bean gum, guar gum, or karaya gum, agar, pectin, carrageenan, gelatin, casein, zein and alginates, carboxypolymethylene, bentonite, magnesium aluminum silicate, polysaccharides, modified starch derivatives and copolymers or mixtures thereof.

Preferably, the polymers may be present in an amount ranging from about 1% to about 35% by weight of the composition.

Suitable channeling agents for use in compositions of the invention, may comprise one or more, but are not limited to sodium chloride, sugars, polyols and the like and mixtures thereof.

Preferably; the channeling agents may be present in an amount ranging from about 0.5% to about 10% by weight of the composition.

Suitable carriers, diluents or fillers for use, in the pharmaceutical composition of the present invention may comprise one or more, but not limited to lactose (for example, spray-dried lactose, α-lactose, β-lactose) lactose available under the trade mark Tablettose, various grades of lactose available under the trade mark Pharmatose or other commercially available forms of lactose, lactitol, saccharose, sorbitol, mannitol, dextrates, dextrins, dextrose, maltodextrin, croscarmellose sodium, microcrystalline cellulose (for example, microcrystalline cellulose available under the trade mark Avicel), hydroxypropylcellulose, L-hydroxypropylcellulose (low substituted), hydroxypropyl methylcellulose (HPMC), methylcellulose polymers (such as, for example, Methocel A, Methocel A4C, Methocel A15C, Methocel A4M), hydroxyethylcellulose, sodium carboxymethylcellulose, carboxymethylene, carboxymethyl hydroxyethylcellulose and other cellulose derivatives, starches or modified starches (including potato starch, corn starch, maize starch and rice starch) and mixtures thereof.

Glidants, anti-adherents and lubricants may also be incorporated in the pharmaceutical composition of the present invention, which may comprise one or more, but not limited to stearic acid and pharmaceutically acceptable salts or esters thereof (for example, magnesium stearate, calcium stearate, sodium stearyl fumarate or other metallic stearate), talc, waxes (for example, microcrystalline waxes) and glycerides, light mineral oil, PEG, silica acid or a derivative or salt thereof (for example, silicates, silicon dioxide, colloidal silicon dioxide and polymers thereof, crospovidone, magnesium aluminosilicate and/or magnesium alumino metasilicate), sucrose ester of fatty acids, hydrogenated vegetable oils (for example, hydrogenated castor oil), or mixtures thereof.

Suitable binders may also present in the pharmaceutical composition of the present invention, which may comprise one or more, but not limited to polyvinyl pyrrolidone (also known as povidone), polyethylene glycol(s), acacia, alginic acid, agar, calcium carragenan, cellulose derivatives such as ethyl cellulose, methyl cellulose, hydroxypropyl cellulose, hydroxypropyl methyl cellulose, sodium carboxymethylcellulose, dextrin, gelatin, gum arabic, guar gum, tragacanth, sodium alginate, or mixtures thereof or any other suitable binder.

Disintegrants may also be present in the pharmaceutical composition of the present invention, which may comprise one or more, but not limited to hydroxylpropyl cellulose (HPC), low density HPC, carboxymethylcellulose (CMC), sodium CMC, calcium CMC, croscarmellose sodium; starches exemplified under examples of fillers and also carboxymethyl starch, hydroxylpropyl starch, modified starch; crystalline cellulose, sodium starch glycolate; alginic acid or a salt thereof, such as sodium alginate or their equivalents and mixtures thereof.

The pharmaceutical composition of the invention can comprise a surfactant, a viscosity builder, a polymer, a carrier, a diluent, a filler, a glidant, an anti-adherent, a lubricant, a binder, a disintegrant, or any combination thereof.

The pharmaceutical composition of the invention preferably comprises a surfactant, a viscosity builder, a polymer, a carrier/diluent/filler, a lubricant/anti-adherent or glidant, a binder and a disintegrant. More preferably, the pharmaceutical composition of the invention comprises a surfactant, a binder, a viscosity builder, a polymer, a carrier and a lubricant.

The fixed dose pharmaceutical composition, according to the present invention, may also optionally be coated, but not limited to seal coating, film coating or a combination thereof.

According to an embodiment of the present invention, pharmaceutical composition may be film coated with, but not limited to, colour mix systems (such as Opadry colour mix systems) and Kollicoat® Protect.

The seal coat may comprise film forming polymeric materials, such as but not limited to, hydroxypropylmethylcellulose, hydroxypropylcellulose, polyvinylpyrrolidone, methylcellulose, carboxymethylcellulose, hypromellose, acacia, gelatin to increase adherence and coherence of the seal coat.

A pharmaceutically acceptable opacifier may also be used in the pharmaceutical composition of the present invention and may comprise one or more, but is not limited to titanium dioxide.

The pharmaceutical composition of the present invention, may further comprise at least one additional active ingredient such as, but not limited to, leukotriene, probenecid, indomethacin, penicillin G, ritonavir, indinavir, saquinavir, furosemide, methotrexate, sulfinpyrazone, interferon, ribavirin, viramidine, valopicitabine, aromatase inhibitor, antiestrogen, anti-androgen, gonadorelin agonist, topoisomerase I inhibitor, topoisomerase II inhibitor, microtubule active agent, alkylating agent, anti-neoplastic, anti-metabolite, platin compound, anti-angiogenic compound, cyclooxygenase inhibitor, bisphosphonate, heparanase inhibitor, telomerase inhibitor, protease inhibitor, matrix metalloproteinase inhibitor, proteasome inhibitor, somatostatin receptor antagonist, anti-leukemic compound, ribonucleotide reductase inhibitor, S-adenosylmethionine decarboxylase inhibitor; ACE inhibitor, antibiotics such as gentamicin, amikacin, tobramycin, ciprofloxacin, levofloxacin, ceftazidime, cefepime, cefpirome, piperacillin, ticarcillin, meropenem, imipenem, polymyxin B, colistin and aztreonam; cyclosporin A, cyclosporin G, rapamycin.

The present invention also provides a method of reducing chronic iron overload by administering a fixed dose pharmaceutical composition comprising deferasirox and deferiprone.

The present invention also provides the use of treating chronic iron overload in thalassemia major patients through chelation therapy by administering fixed dose pharmaceutical composition comprising deferasirox and deferiprone.

The following example is for the purpose of illustration of the invention only and is not intended in any way to limit the scope of the present invention.

### Example

| | | 500 mg/250 mg | 250 mg/125 mg | 375 mg/125 mg |
|---|---|---|---|---|
| Sr. No. | Ingredients | (mg/tablet) | (mg/tablet) | (mg/tablet) |
| I] | Binder solution | | | |
| 1 | Deferasirox | 500.00 | 250.00 | 375.00 |
| 2 | Deferiprone | 250.00 | 125.00 | 125.00 |
| 3 | Docusate sodium | 10.00 | 5.00 | 7.5 |
| 4 | Hydroxypropylmethylcellulose | 100.00 | 50.00 | 75.00 |
| 5 | Sodium lauryl sulphate | 24.00 | 12.00 | 18.00 |
| 6 | Sucrose | 150.00 | 75.00 | 112.5 |
| 7 | Purified water | q.s. | q.s. | q.s. |
| II] | Dry mix | | | |
| 8 | Lactose monohydrate | 200.00 | 100.00 | 125.00 |
| 9 | Microcrystalline cellulose | 197.00 | 98.5 | 135.25 |
| 10 | Crospovidone | 50.00 | 25.00 | 37.5 |
| III] | Lubrication | | | |
| 11 | Crospovidone | 50.00 | 25.00 | 37.5 |
| 12 | Sodium chloride | 60.00 | 30.00 | 45.00 |
| 13 | Magnesium stearate | 9.00 | 4.5 | 6.75 |
| | Total | 1600.00 | 800.00 | 1100.00 |

### Process 1:

1. Docusate sodium, HPMC, sodium lauryl sulphate and sucrose were solubilized.

2. Deferasirox and Deferiprone were added in the solution obtained in step (1), homogenized and then nanomilled.

3. Nanomilled slurry obtained in step (2) was adsorbed by spraying on lactose monohydrate, microcrystalline cellulose and crospovidone mixture to produce granules.

4. Granules so obtained were dried, sized, lubricated and compressed into tablets.

### Process 2:

### A) Deferasirox Granules

1. Docusate sodium, HPMC, sodium lauryl sulphate and sucrose were solubilized.

2. Deferasirox was added in the solution obtained in step (1), homogenized and then nanomilled.

3. Nanomilled slurry obtained in step (2) was adsorbed by spraying on lactose monohydrate, microcrystalline cellulose and crospovidone mixture to produce granules.

### B) Deferiprone Granules

1. Docusate sodium, HPMC, sodium lauryl sulphate and sucrose were solubilized.

2. Deferiprone was added in the solution obtained in step (1), homogenized and then nanomilled.

3. Nanomilled slurry obtained in step (2) was adsorbed by spraying on lactose monohydrate, microcrystalline cellulose and crospovidone mixture to produce granules.

### C) Tablet Compression

1. Deferasirox and Deferiprone granules so obtained in were dried, sized, lubricated and compressed into tablets.

### Process 3:

### A) Deferasirox Granules

1. Docusate sodium, HPMC, sodium lauryl sulphate and sucrose were solubilized.

2. Deferasirox was added in the solution obtained in step (1), homogenized and then nanomilled.

### B) Deferiprone Granules

1. Docusate sodium, HPMC, sodium lauryl sulphate and sucrose were solubilized.

2. Deferiprone was added in the solution obtained in step (1), homogenized and then nanomilled.

### C) Granulation and Tablet Compression

1, Nanomilled slurry of Deferasirox and Deferiprone was adsorbed by spraying on lactose monohydrate, microcrystalline cellulose and crospovidone mixture to produce granules.

2. Granules so obtained in were dried, sized, lubricated and compressed into tablets.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “an excipient” includes a single excipient as well as two or more different excipients, and the like.

1. A fixed dose pharmaceutical composition comprising at least two iron chelating agents and optionally one or more pharmaceutically acceptable excipients.

2. A fixed dose pharmaceutical composition according to claim 1, comprising at least two iron chelating agents in the form of a pharmaceutically acceptable derivative thereof.

3. A fixed dose pharmaceutical composition according to claim 2, wherein the pharmaceutically acceptable derivative thereof is a salt, solvate, complex, hydrate, isomer, ester, tautomer, anhydrate, enantiomer, polymorph or prodrug.

4. A fixed dose pharmaceutical composition according to claim 1, wherein the at least two iron chelating agents comprise deferasirox and deferiprone.

5. A fixed dose pharmaceutical composition according to claim 1, wherein deferasirox and deferiprone are in the ratio of 1:1.5-5.

6. A fixed dose pharmaceutical composition according to claim 1, wherein the composition is in an oral, parenteral or topical dosage form.

7. A fixed dose pharmaceutical composition according to claim 1, wherein the composition is in a form of a tablet, coated tablet, powders, powders for reconstitution, pellets, beads, mini-tablets, multilayer tablets, bilayered tablets, tablet in tablet, pills, micro-pellets, small tablet units, MUPS, disintegrating tablets, dispersible tablets, granules, and microspheres, multiparticulates, capsules (filled with powders, powders for reconstitution, pellets, beads, mini-tablets, pills, micro-pellets, small tablet units, MUPS, orally disintegrating MUPS, disintegrating tablets, dispersible tablets, granules, sprinkles, microspheres and multiparticulates), sachets (filled with powders, powders for reconstitution, pellets, beads, mini-tablets, pills, micro-pellets, small tablet units, MUPS, disintegrating tablets, dispersible tablets, modified release tablets or capsules, effervescent granules, granules, sprinkles microspheres and multiparticulates) and sprinkles.

8. A fixed dose pharmaceutical composition according to claim 7, wherein the solid dosage form is a single layer or a bilayer tablet or a multilayer tablet.

9. A fixed dose pharmaceutical composition according to claim 7, wherein the solid dosage form is a dispersible tablet.

10. A fixed dose pharmaceutical composition according to claim 1, wherein the one or more pharmaceutically acceptable excipients comprise a surfactant.

11. A fixed dose pharmaceutical composition according to claim 1, wherein the one or more pharmaceutically acceptable excipients comprise a viscosity builder.

12. A fixed dose pharmaceutical composition according to claim 1, wherein the one or more pharmaceutically acceptable excipients comprise a polymer.

13. A fixed dose pharmaceutical composition according to claim 1, wherein the one or more pharmaceutically acceptable excipients comprise a surfactant, a viscosity builder, a polymer, a carrier, a diluent, a filler, a glidant, an anti-adherent, a lubricant, a binder, a disintegrant, or any combination thereof.

14. A fixed dose pharmaceutical composition according to claim 1, wherein the pharmaceutical composition further comprises an additional active pharmaceutical ingredient such as leukotriene, probenecid, indomethacin, penicillin G, ritonavir, indinavir, saquinavir, furosemide, methotrexate, sulfinpyrazone, interferon, ribavirin, viramidine, valopicitabine, aromatase inhibitor, antiestrogen, anti-androgen, gonadorelin agonist, topoisomerase I inhibitor, topoisomerase II inhibitor, microtubule active agent, alkylating agent, anti-neoplastic, anti-metabolite, platin compound, anti-angiogenic compound, cyclooxygenase inhibitor, bisphosphonate, heparanase inhibitor, telomerase inhibitor, protease inhibitor, matrix metalloproteinase inhibitor, proteasome inhibitor, somatostatin receptor antagonist, anti-leukemic compound, ribonucleotide reductase inhibitor, S-adenosylmethionine decarboxylase inhibitor; ACE inhibitor, antibiotics such as gentamicin, amikacin, tobramycin, ciprofloxacin, levofloxacin, ceftazidime, cefepime, cefpirome, piperacillin, ticarcillin, meropenem, imipenem, polymyxin B, colistin and aztreonam; cyclosporin A, cyclosporin G, rapamycin or combinations thereof.

15. A fixed dose pharmaceutical composition according to claim 1 for use in the treatment of chronic iron overload.

16. Use of a fixed dose pharmaceutical composition according to claim 1 in the manufacture of a medicament for treating chronic iron overload.

17. A method of treating chronic iron overload wherein the method comprises administering a fixed dose pharmaceutical composition according to claim 1.

18. A process for preparing a fixed dose pharmaceutical composition according to claim 1, wherein the process comprises mixing at least two chelating agents with one or more pharmaceutically acceptable excipients; and forming the fixed dose pharmaceutical composition.

19. A process according to claim 18, wherein the process comprises:
(1) homogenizing deferasirox and/or deferiprone and at least one excipient to produce a homogenized dispersion;
(2) milling the said homogenized dispersion to produce a slurry comprising deferasirox and/or deferiprone particles having an average particle size of less than or equal to about 2000 nm.; and
(3) processing the slurry to obtain the dosage form.

20. (canceled)
